(19) United States
(12) Patent Application Publication
(10) Pub. No.: US 2022/0307021 A1
(43) Pub. Date: Sep. 29, 2022

(54) A NEUROPILIN ANTAGONIST IN COMBINATION WITH A P38ALPHA-KINASE INHIBITOR FOR THE TREATMENT OF CANCER

(71) Applicants: INSERM (Institut National de la santé et de la Recherche Médicale), Paris (FR); Université de Paris, Paris (FR); Centre National de la Recherche Scientifique (CNRS), Paris (FR); Assistance Publique-Hôpitaux de Paris (APHP), Paris (FR); Fondation Imagine, Paris (FR); Conservatoire National des Arts et Métiers (CNAM), Paris (FR)

(72) Inventors: Yves LEPELLETIER, Paris (FR); Matthieu MONTES, Paris (FR); Luc DEMANGE, Paris (FR); François RAYNAUD, Fontenay-aux-Roses (FR); Rachel RIGNAULT-BRICARD, Paris (FR); Olivier HERMINE, Paris (FR); Nicolas LOPEZ, Paris (FR)

(21) Appl. No.: 17/614,437

(22) PCT Filed: Jun. 3, 2020
(86) PCT No.: PCT/EP2020/065369

§ 371 Date: Nov. 26, 2021

(30) Foreign Application Priority Data
Jun. 4, 2019 (EP) 19305719.7

(51) Int. Cl. C12N 15/113; A61P 35/00; C07K 16/28

(52) U.S. Cl. CPC C12N 15/113; A61P 35/00; C07K 16/2866; C07K 2317/76; C07K 2317/73; C12N 2310/11

(57) ABSTRACT

Neuropilin-1 is henceforth a relevant target in cancer treatment, however way-of-action is remains partly elusive and the development of small inhibitory molecules is therefore required for its study. Here, the inventors report that two neuropilin small-sized antagonists (NRPa-47, NRPa-48), VEGF-A165/NRP-1 binding inhibitors, are able to decrease VEGF-Rs phosphorylation and to modulate their downstream cascades in triple negative breast cancer cell line (MDA-MB-231). In particular, the inventors showed for the first time, how NRPa may altered tumor cell signaling and contributed in the down-modulation of the cancer therapeutic key factor p38α-kinase phosphorylation. More importantly, the association of NRPa with a p38α inhibitor leads to additional and/or synergistic effect of these drugs (depending of the dose used) for significantly reducing breast cancer cell proliferation Thus, the efficient association of NRPa and p38α-kinase inhibitors are thus credible for the treatment of cancer.

### FIELD OF THE INVENTION

The present invention relates to the field of medicine, in particular of oncology.

### BACKGROUND OF THE INVENTION

Neuropilin-1 (NRP-1) and neuropilin-2 (NRP-2) are transmembrane type I glycoproteins sharing 44% sequence homology [1]. Initially, neuropilins (NRPs) were identified as neuronal receptor of specific secreted members of semaphorin III family involved in guidance and in repulsion axonal [2]. Neuropilins are multifunctional non-tyrosine kinase receptors for some members of VEGF (Vascular Endothelial Growth Factor) family members. VEGF-A₁₆₅, a VEGF-A spliced form, which up-regulation is reported in several tumor tissues, is considered as one of the most efficient pro-angiogenic factors. VEGF-A₁₆₅ binds to structurally related tyrosine kinase receptors such as VEGF-R1 (Flt-1), VEGF-R2 (Flk-2) and to NRPs, co-receptors lacking cytosolic catalytic activity [3, 1]. In many cancers, expression of one or both NRPs has been correlated with tumor progression and/or poor prognosis (see for review) [4, 5]. Through their direct interactions with VEGF-Rs, NRPs have rapidly emerged as key regulators of angiogenesis and tumor progression.

These identified protein-protein interactions triggering angiogenesis processes have led to the development of extra-cellular VEGF-trap, such as monoclonal antibodies (e.g. Avastin®), aptamer (e.g. Macugen®) and to small molecules targeting the intracellular kinase activity of its tyrosine kinase receptors (e.g. Sutent®). Nevertheless, the existence of different pathways involved in angiogenesis, and the emergence of therapeutic resistance in patients associated with a generally poor response exerts the necessity to develop new anti-angiogenic strategies.

The drug development against the emerging NRP target brought newest tools such as antibodies [1, 6, 7], peptides (A7R, EG3287, NRP-1 trans-membranar peptides) [8-12] and peptidomimetic (EG00229) [13]. Recently, new approaches highlighted the interest of small inhibitory molecules to decrease VEGF binding to NRP, tumor growth in vivo and in vitro [14, 15]. In this field, we are the first research team, which develop a fully non-peptidic inhibitory molecule so-called neuropilin antagonist (NRPa) [15]. However, the molecular mechanism by which NRPs modulate cancer progression are still poorly understood. NRPa should provide additional data for the rational knowledge of the cell signaling involved in tumor development and survival.

### SUMMARY OF THE INVENTION

As defined by the claims, the present invention relates to a neuropilin antagonist in combination with a p38α-kinase inhibitor for the treatment of cancer.

### DETAILED DESCRIPTION OF THE INVENTION

Neuropilin-1 is henceforth a relevant target in cancer treatment, however is way-of-action remains partly elusive and the development of small inhibitory molecules is therefore required for its study. Here, the inventors report that two neuropilin small-sized antagonists (NRPa-47, NRPa-48), VEGF-A₁₆₅/NRP-1 binding inhibitors, are able to decrease VEGF-Rs phosphorylation and to modulate their downstream cascades in triple negative breast cancer cell line (MDA-MB-231). Nevertheless, NRPa exert a divergent pathway regulation of MAPKs phosphorylation such as JNK-1/-2/-3, ERK-1/-2 and p38β/γ/δ-kinases as well as their respective downstream targets. However, NRPa-47 and NRPa-48 apply a common down-regulation of the p38α-kinase phosphorylation and their downstream targets emphasizing its central regulating role. More importantly, none of the 40-selected kinases, including SAPK2a/p38α are affected in vitro by NRPa, strengthened their specificity. Taking together, NRPa induced cell death by the down-modulation of pro-apoptotic and anti-apoptotic proteins, cell death receptors and adaptors, heat shock proteins (HSP-27/-60/-70), cell cycle proteins (p21, p27, phospho-RAD17) and transcription factors (p53, HIF-1α). In conclusion, we showed for the first time, how NRPa may altered tumor cell signaling and contributed in the down-modulation of the cancer therapeutic key factor p38α-kinase phosphorylation. Thus, the efficient association of NRPa and p38α-kinase inhibitors is thus credible for the treatment of cancer.

A further object of the present invention relates to a method of treating cancer in a patient in need thereof comprising administering to the patient a therapeutically effective combination comprising at least one neuropilin antagonist and at least one p38α-kinase inhibitor.

As used herein, the term “subject” or “patient” refers to a mammal, preferably a human. Examples of non-human mammal include a pet such as a dog, a cat, a domesticated pig, a rabbit, a ferret, a hamster, a mouse, a rat and the like; a primate such as a chimp, a monkey, and the like; an economically important animal such as cattle, a pig, a rabbit, a horse, a sheep, a goat.

As used herein, the term “cancer” has its general meaning in the art and includes, but is not limited to, hematopoietic cancers (e.g. blood borne tumors) and non-hematopoietic cancers (e.g. solid tumors). The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the method of the present invention is particularly suitable for the treatment of breast cancer and in particular for the treatment of triple negative breast cancer. As used herein the expression “triple negative breast cancer” has its general meaning in the art and means that said breast cancer lacks receptors for the hormones estrogen (ER-negative) and progesterone (PR-negative), and for the protein HER2.

In some embodiments, the cancer has previously screened as “neuropilin positive”, i.e. the cancer cells express a neuropilin protein. Said expression may be assessed in the tumor by any routine method known in the art, such as immunohistochemistry (IHC), immunofluorescence, mass spectrometry, RT-PCR, fluorescence in situ hybridization (FISH), chromogenic in situ hybridization (CISH), silver in situ hybridization (SISH)) or comparative genomic hybridization (CGH), RNAscope . . . .

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]). In particular, in the case of AMLs, maintenance therapy may eradicate clinically invisible minimal residual disease.

As used herein, the term “neuropilin” or “NRP” has its general meaning in the art and refers to a transmembrane glycoprotein that typically consists of five domains: three extracellular domains (a1 a2, b1, b2 and c), a transmembrane domain and a cytoplasmic domain. There are two neuropilin members: neuropilin-1 (NRP-1) and neuropilin-2 (NRP-2) that are share 44% sequence homology. Neuropilins are multifunctional non-tyrosine kinase receptors for some members of VEGF (Vascular Endothelial Growth Factor) family members, including VEGF-A₁₆₅.

As used herein, the term “neuropilin antagonist” refers to a molecule that partially or fully blocks, inhibits, or neutralizes a biological activity or expression of a neuropilin protein. A neuropilin antagonist can be a molecule of any type that interferes with the signaling associated with at least one or more neuropilin family members (e.g. NRP-1 or NRP-2) in a cell, for example, either by decreasing transcription or translation of neuropilin-encoding nucleic acid, or by inhibiting or blocking neuropilin polypeptide activity, or both. Examples of neuropilin antagonists include, but are not limited to, antisense polynucleotides, interfering RNAs, catalytic RNAs, RNA-DNA chimeras, neuropilin-specific aptamers, anti-neuropilin antibodies, neuropilin-binding fragments of anti-neuropilin antibodies, neuropilin-binding small molecules, neuropilin-binding peptides, and other polypeptides that specifically bind neuropilin (including, but not limited to, neuropilin-binding fragments of one or more neuropilin ligands, optionally fused to one or more additional domains), such that the interaction between the neuropilin antagonist and neuropilin results in a reduction or cessation of neuropilin activity or expression. In particular, neuropilin antagonist inhibits the interaction between a neuropilin protein (e.g. NRP-1) and its partners, in particular VEGF-A₁₆₅.

Neuropilin antagonists are well known in the art and typically include those describe in:
Liu W Q, Megale V, Borriello L, Leforban B, Montès M, Goldwaser E, Gresh N, Piquemal J P, Hadj-Slimane R, Hermine O, Garbay C, Raynaud F, Lepelletier Y, Demange L. Synthesis and structure-activity relationship of non-peptidic antagonists of neuropilin-1 receptor. Bioorg Med Chem Lett. 2014 Sep. 1; 24(17):4254-9.
Tymecka D, Puszko A K, Lipiński PFJ, Fedorczyk B, Wilenska B, Sura K, Perret G Y, Misicka A. Branched pentapeptides as potent inhibitors of the vascular endothelial growth factor 165 binding to Neuropilin-1: Design, synthesis and biological activity. Eur J Med Chem. 2018 Oct. 5; 158:453-462.
Liu W Q, Lepelletier Y, Montès M, Borriello L, Jarray R, Grépin R, Leforban B, Loukaci A, Benhida R, Hermine O, Dufour S, Pagès G, Garbay C, Raynaud F, Hadj-Slimane R, Demange L. NRPa-308, a new neuropilin-1 antagonist, exerts in vitro anti-angiogenic and anti-proliferative effects and in vivo anti-cancer effects in a mouse xenograft model. Cancer Lett. 2018 Feb. 1; 414:88-98.
Borriello L, Montès M, Lepelletier Y, Leforban B, Liu W Q, Demange L, Delhomme B, Pavoni S, Jarray R, Boucher J L, Dufour S, Hermine O, Garbay C, Hadj-Slimane R, Raynaud F. Structure-based discovery of a small non-peptidic Neuropilins antagonist exerting in vitro and in vivo anti-tumor activity on breast cancer model. Cancer Lett. 2014 Jul. 28; 349(2):120-7.
Getz J A, Cheneval O, Craik D J, Daugherty P S. Design of a cyclotide antagonist of neuropilin-1 and -2 that potently inhibits endothelial cell migration. ACS Chem Biol. 2013; 8(6):1147-54.
Jia H, Bagherzadeh A, Hartzoulakis B, Jarvis A, Löhr M, Shaikh S, Agil R, Cheng L, Tickner M, Esposito D, Harris R, Driscoll P C, Selwood D L, Zachary I C. Characterization of a bicyclic peptide neuropilin-1 (NP-1) antagonist (EG3287) reveals importance of vascular endothelial growth factor exon 8 for NP-1 binding and role of NP-1 in KDR signaling. J Biol Chem. 2006 May 12; 281(19):13493-502.
A. Starzec, P. Ladam, R. Vassy, S. Badache, N. Bouchemal, A. Navaza, C. H. du Penhoat and G. Y. Perret, Structure-function analysis of the antiangiogenic ATWLPPR peptide inhibiting VEGF(165) binding to neuropilin-1 and molecular dynamics simulations of the ATWLPPR/neuropilin-1 complex, Peptides 28 (2007) 2397-402.
A. Novoa, N. Pellegrini-Moise, D. Bechet, M. Barberi-Heyob and Y. Chapleur, Sugar-based peptidomimetics as potential inhibitors of the vascular endothelium growth factor binding to neuropilin-1, Bioorg. Med. Chem 18. (2010) 3285-98.
C. Nasarre, M. Roth, L. Jacob, L. Roth, E. Koncina, A. Thien, G. Labourdette, P. Poulet, P. Hubert, G. Crémel, G. Roussel, D. Aunis, D. Bagnard, Peptide-based interference of the transmembrane domain of neuropilin-1 inhibits glioma growth in vivo, Oncogene 29 (2010) 2381-92.

In some embodiments, the neuropilin antagonist is an antibody that specifically binds to a neuropilin (e.g. NRP-1 or NRP-2) and neutralizes its activity to activate neuropilin signalling pathway, and in particular inhibits the binding neuropilin and VEGF-A₁₆₅. In some embodiments, the antibody binds to an extracellular domain of neuropilin. In some embodiments, the antibody binds to the domain c of NRP-1. Examples of antibodies that are neuropilin antagonists include those described in WO2011/143408 that described in particular the anti-NRP-1 antibody MNRP1685A.

As used herein, the term “antibody” as includes but is not limited to polyclonal, monoclonal, humanized, chimeric, Fab fragments, Fv fragments, F(ab′) fragments and F(ab′)2 fragments, as well as single chain antibodies (scFv), fusion proteins and other synthetic proteins which comprise the antigen-binding site of the antibody. Antibodies can be made by the skilled person using methods and commercially available services and kits known in the art. Methods of preparation of monoclonal antibodies are well known in the art and include hybridoma technology and phage display technology. Further antibodies suitable for use in the present disclosure are described, for example, in the following publications: Antibodies A Laboratory Manual, Second edition. Edward A. Greenfield. Cold Spring Harbor Laboratory Press (Sep. 30, 2013); Making and Using Antibodies: A Practical Handbook, Second Edition. Eds. Gary C. Howard and Matthew R. Kaser. CRC Press (Jul. 29, 2013); Antibody Engineering: Methods and Protocols, Second Edition (Methods in Molecular Biology). Patrick Chames. Humana Press (Aug. 21, 2012); Monoclonal Antibodies: Methods and Protocols (Methods in Molecular Biology). Eds. Vincent Ossipow and Nicolas Fischer. Humana Press (Feb. 12, 2014); and Human Monoclonal Antibodies: Methods and Protocols (Methods in Molecular Biology). Michael Steinitz. Humana Press (Sep. 30, 2013)).

In some embodiments, the neuropilin antagonist is a small molecule, such as a small organic molecule, which typically has a molecular weight less than 5,000 kDa.

Examples of small molecules that are neuropilin antagonists include those described in WO2012156289 that are:

### N-[5-(1H-benzimidazol-2-yl)-2-methylphenyl]-N′-(2,3-dihydro-1,4-benzodioxin-6-ylcarbonyl)thiourea (Also Named NRPa-47)

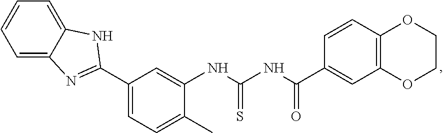 [SMILES: Cc1ccc(-c2nc3ccccc3[nH]2)cc1NC(=S)NC(=O)c1ccc2c(c1)OCCO2]

### N-[3-(1H-benzimidazol-2-yl)phenyl]-N′-(2,3-dihydro-1,4-benzodioxin-6-ylcarbonyl)thiourea (Also Named NRPa-48)

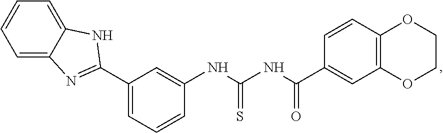 [SMILES: O=C(NC(=S)Nc1cccc(-c2nc3ccccc3[nH]2)c1)c1ccc2c(c1)OCCO2]

and/or

### N-[3-(1H-benzimidazol-2-yl)phenyl]-N′-(1,3-benzodioxol-5-ylcarbonyl)thiourea

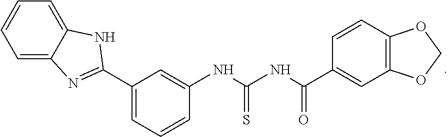 [SMILES: O=C(NC(=S)Nc1cccc(-c2nc3ccccc3[nH]2)c1)c1ccc2c(c1)OCO2]

or their salts and esters, and mixtures thereof.

Another example includes N-(2-ethoxyphenyl)-4-methyl-3-(N-(p-tolyl)sulfamoyl)benzamide that has been described in WO2015004212 and having the formula of

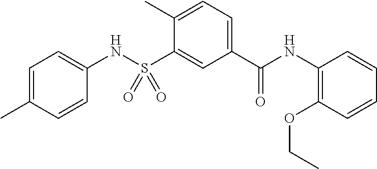 [SMILES: CCOc1ccccc1NC(=O)c1ccc(C)c(S(=O)(=O)Nc2ccc(C)cc2)c1]

In some embodiments, the neuropilin antagonist is an inhibitor of neuropilin expression.

An “inhibitor of expression” refers to a natural or synthetic compound that has a biological effect to inhibit the expression of a gene. In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of NRP-1 mRNA by binding thereto and thus preventing protein translation or increasing mRNA degradation, thus decreasing the level of NRP-1, and thus activity, in a cell. For example, antisense oligonucleotides of at least about 15 bases and complementary to unique regions of the mRNA transcript sequence encoding NRP-1 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. NRP-1 gene expression can be reduced by contacting a patient or cell with a small double stranded RNA (dsRNA), or a vector or construct causing the production of a small double stranded RNA, such that NRP-1 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing NRP-1. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. In a particular embodiment, the endonuclease is CRISPR-cas. In some embodiment, the endonuclease is CRISPR-cas9, which is from Streptococcus pyogenes. The CRISPR/Cas9 system has been described in U.S. Pat. No. 8,697,359 B1 and US 2014/0068797. In some embodiment, the endonuclease is CRISPR-Cpf1, which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

As used herein, the term “p38α-kinase” has its general meaning in the art and refers to a member of the p38 mitogen-activated protein kinases (MAPKs). The p38 MAPK family includes four members, p38-α (MAPK14), p38-β (MAPK11), p38-γ (MAPK12/ERK6), and p38-δ (MAPK13/SAPK4), which are involved in a signaling cascade that controls cellular response.

As used herein, the term “p38α-kinase inhibitor” refers to a molecule that partially or fully blocks, inhibits, or neutralizes a biological activity or expression of a p38α protein. Suitable inhibitor molecules specifically include antagonist antibodies or antibody fragments, fragments or amino acid sequence variants of native polypeptides, peptides, antisense oligonucleotides, small organic molecules, recombinant proteins or peptides, etc. A p38α-kinase inhibitor can be a molecule of any type that interferes with the signaling associated with at least p38-α, for example, either by decreasing transcription or translation of p38-α encoding nucleic acid, or by inhibiting or blocking p38-α kinase activity, or both. In some examples, a p38α-kinase inhibitor is an agent that interferes with the signaling associated with p38-α. Examples of p38α-kinase inhibitors include, but are not limited to, antisense polynucleotides, interfering RNAs, catalytic RNAs, RNA-DNA chimeras, p38-α-specific aptamers, anti-p38α antibodies, p38α-binding fragments of anti-p38α antibodies, p38α-binding small molecules, p38α-binding peptides, and other polypeptides that specifically bind p38α (including, but not limited to, p38α-binding fragments of one or more p38α ligands, optionally fused to one or more additional domains), such that the interaction between the p38α-kinase inhibitor and p38α results in a reduction or cessation of p38α kinase activity or expression. For example, a desirable p38α-kinase inhibitor for use in certain of the methods herein is a p38α-kinase inhibitor that binds p38-α and blocks p38α signaling, e.g., without affecting or minimally affecting any of the other member of the p38 MAPK family, for example, binding p38-β, p38-γ, and/or p38-δ. It will be appreciated that p38α-kinase inhibitors described herein may be strong inhibitors of p38α. For example, the p38α-kinase inhibitor has a binding inhibitory activity (IC50 value) for p38α of 1000 μM or less, 1000 nM or less, 100 nM or less, 10 nM or less, or especially 1 nM or less. In another example, the p38α-kinase inhibitor has a binding inhibitory activity (IC50 value) for p38α of between 1000 μM and 1 nM, between 1000 μM and 10 nM, between 1000 μM and 100 nM, between 1000 μM and 1000 nM, between 1000 nM and 1 nM, between 1000 nM and 10 nM, between 1000 nM and 100 nM, between 100 nM and 10 nM, between 100 nM and 1 nM, or between 10 nM and 1 nM.

In particular, the p38α-kinase inhibitor is a small molecule, such as a small organic molecule, which typically has a molecular weight less than 5,000 kDa. Inhibitors of p38α include, but are not limited to, ARRY-371797 (ARRY-797; Array BioPharma Inc.), ARRY-614 (pexmetinib; Array BioPharma Inc. or Selleckchem), AZD-7624 (AstraZeneca Plc), LY-2228820 (ralimetinib dimesylate; Eli Lilly and Co. or Selleckchem), LY-3007113 (Eli Lilly and Co.), FX005 (Flexion Therapeutics Inc.), GSK610677 (GlaxoSmithKline Plc), GW856553 (GW856553X; losmapimod; GlaxoSmithKline Plc or Selleckchem), SB-681323 (dilmapimod; GlaxoSmithKline Plc), KC706 (Kemia Inc.), UR-13870 (Palau Pharma S.A.), PF-03715455 (PF-3715455; Pfizer Inc.), VX-745 (Vertex Pharmaceuticals Inc. or Selleckchem), SCID-469 (talmapimod; Scios Inc.), PH-797804 (Pfizer or Selleckchem), VX-702 (Selleckchem), SB-202190 (FHPI; Selleckchem), SB-203580 (Selleckchem), SB-239063, BIRB-796 (doramapimod; Selleckchem), BMS-582949, and pamapimod.

In some embodiments, the p38α-kinase inhibitor is an inhibitor of p38α-kinase expression.

As used herein, the term “combination” is intended to refer to all forms of administration that provide a first drug together with a further (second, third . . . ) drug. The drugs may be administered simultaneous, separate or sequential and in any order. Drugs administered in combination have biological activity in the patient to which the drugs are delivered. Within the context of the invention, a combination thus comprises at least two different drugs, and wherein one drug is at least one neuropilin antagonist and wherein the other drug is at least one p38α-kinase inhibitor. In some instance, the combination of the present invention results in the synthetic lethality of cancer cells.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. A therapeutically effective amount of a therapeutic compound may decrease tumour size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, the drug of the present invention is administered to the subject in the form of a pharmaceutical composition, which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials.

The invention will be further illustrated by the following figures and examples. However, these examples and figures should not be interpreted in any way as limiting the scope of the present invention.

### FIGURES

FIG. 1: NRPa-47 induces VEGF downstream signaling modification. Histograms show pixel intensity of untreated (ct, white histograms), NRPa-47-treated MDA-MB-231 at 10 (histograms with bands) and 60 minutes (black histograms) of p38α/p38β/p38γ/p38δ. Histograms represent means±SD of previous representative selected experiment analyzed using Image-J software to quantify pixel intensity. (□, P<0.05; **, P<0.01; ***, P<0.001; NS=not significant).

FIG. 2: NRPa-48 derived-NRPa-47 mechanism of action on downstream VEGF signaling. Histograms show pixel intensity of untreated (ct, white histograms), NRPa-48-treated (IC₅₀=0.4 μM) MDA-MB-231 at 10 (histograms with bands) and 60 minutes (black histograms) of p38α/p38β/p38γ/p38δ. Histograms represent means±SD of previous representative selected experiment analyzed using Image-J software to quantify pixel intensity. (□, P<0.05; **, P<0.01; ***, P<0.001; NS=not significant).

FIG. 3: NRPa-47 and NRPa-48 Protein kinase profiling: Protein kinase profiling of NRPa-47 (A) and NRPa-48 (B) was performed at 1 μM on a large selection of 40 kinases including Neuroplin-1 co-receptors and related biochemical kinase signaling observed during this study.

FIG. 4: NRPa/Ralimetinib® association increased anti-breast cancer cell proliferation. A large concentration range of Ralimetinib® is tested alone or in association with NRPa (IC₅₀) (A) or sub-optimal NRPa IC50 (0.1 μM) (B) on MDA MB 231 cells proliferation. NRPa IC₅₀/Ralimetinib® association showed additive effect (AE) at high Ralimetinib® concentration and synergistic effect (SE) at low Ralimetinib® concentration (A). Sub-optimal NRPa IC₅₀/Ralimetinib® association showed additive effect (AE) at high Ralimetinib® concentration and synergistic effect (SE) at low Ralimetinib® concentration. Data represent means±SD of 3 separate experiments, each. (NS: Not significant)

### EXAMPLE

Material & Methods

2.1—Chemical Synthesis of Compound:

Chemical reagents and solvents were purchased from Sigma Aldrich, Fluka and Carlo Erba. NRPa-47 and NRPa-48 has been synthesized and characterized as previously reported by us [16].

2.2—Total RNA Preparation and RT-PCR:

MDA-MB-231 RNA was extracted with NucleoSpinRNA II kit (Macherey-Nagel, France) and quantified using Nanodrop (ND-1000 spectrophotometer). 1 μg of each RNA sample was reverse-transcripted into cDNA using iScript cDNA Synthesis Kit (Bio-Rad, France) following the manufacturer's instructions. PCR amplification was performed in reaction mixture (25 μL) containing 200 μM of each dNTP, 1 μg of cDNA, 1 μM of primers and 0.625 U of GoTaq DNA Polymerase (Promega, France) with 45 s of denaturation at 95° C., 45 s of annealing at 60° C. and 1 min of extension at 72° C. for 30 cycles. PCR products were separated by 1% agarose gel electrophoresis, stained with ethidium bromide (Sigma, Germany) and analyzed using Gel Doc 2000 System (Bio-Rad, France).

2.3—Proteome Profiler Arrays:

MDA-MB-231 cells were incubated in the presence or absence of NRPa-47 or NRPa-48 compounds (IC₅₀) and protein lysates were prepared and quantified as previously described [17, 18]. Biochemical signaling detection was evaluated by using human proteome profiler array (human phosphokinase array and human apoptosis array) according to the manufacturer's instructions (R&D systems, France). Briefly, capture and control antibodies were spotted in duplicate on nitrocellulose membranes. Cellular extracts were incubated overnight on membrane, washed to remove unbound proteins, followed by incubation with a cocktail of biotinylated detection antibodies. Streptavidin-HRP and chemiluminescent detection reagents were applied, and the signal intensity corresponding to the amount of protein bound was measured at each capture spot using ImageJ software.

2.4—Cell Culture Conditions.

The human aggressive and metastatic estrogenR-/progesteroneR-/Her2-triple negative breast cancer cell line (MDA-MB-231) purchased from the ATCC (Molsheim France) were plated in 200 μL/well in 96-well plates at 10.10³ cells/well and were treated or not with NRPa-47 (IC₅₀), NRPa-48 (IC₅₀), 5-FU®, Oxaliplatin® and Ralimetinib® alone or in combination at different concentrations. WST-1 (Roche®, France) was added for 1-2 h, then Optical Density was analyzed with a microplate reader (Microplate Manager 5.2, Bio-Rad) at 490 nm to determine the cell viability. For each compound, the IC50 value was determined from a sigmoid dose-response curve using Graph-Pad Prism (GraphPad Software, San Diego, USA).

2.5—VEGF-R Kinase Assay:

The cells were cultured in the presence of NRPa-47 or NRPa-48 at their IC50 during 5 to 60 min and then the MDA-MB-231 lysates was used to detect total VEGF-R1 and VEGF-R2 tyrosine-phosphorylation using ELISA assay (R&D system).

2.6—Molecular Docking:

The binding site has been defined at 4 Å around the co crystallized tuftsin bound to NRP-1 (PDB code 2ORZ) [19]. Consensus molecular docking was performed using Surflex dock v2.5. [20] and ICM-VLS-v3.4. [21] Surflex dock is based on a modified Hammerhead fragmentation/reconstruction algorithm to dock compounds flexibly into the binding site. The query molecule is decomposed into rigid fragments that are superimposed on the Surflex Protocol, i.e., molecular fragments covering the entire binding site. The docking poses were evaluated by an empirical scoring function. ICM is based on Monte Carlo simulations in internal coordinates to optimize the position of molecules using a stochastic global optimization procedure combined with pseudo-Brownian positional/torsional steps and fast local gradient minimization. The docking poses were evaluated using the ICM VLS empirical scoring function.

2.7—Protein Kinase Profiling:

NRPa-47 and NRPa-48 specificity profiling assays were carried out at Eurofins Pharma Discovery Services (Dundee, UK) for Protein Kinase Profiling against a selected panel of 40 protein kinases. Results of protein kinases assayed at 1 μM of each NRPa are presented as a percentage of kinase activity in DMSO control reactions.

2.8—In Vivo Xenografted-Tumor Mouse Model:

Protocol was approved by the INSERM Institutional Care and Use Committee according to the European Communities Council Directive. NOD/scid/IL-2Rγ−/− (NOG) female mice were bred and housed in pathogen-free conditions in accordance with the Federation of European Animal Associations (FELSA) guidelines. MDA-MB-231 cells were washed twice in PBS and resuspended in DMEM. Subsequently, cells were injected subcutaneously into NOG mice (6-7 weeks old) at the concentration of 2.10⁶ cells/200 μL. Mice were randomly divided into different groups (10 mice/group). Mice then received using force-feeding NRPa-48 (50 mg/kg, respectively) or vehicle every three days for 39 days. Tumor growth and body weight were measured every three days during the treatment. Mice were weighed regularly to assess the toxicity of the treatment and the tumors were measured with calipers (width×width×length×Pi/6) to determine growth.

2.9—Statistical Analysis:

Data are expressed as the arithmetic mean+/−SD of at least three different experiments. The statistical significance of results was evaluated by ANOVA, with probability values *p<0.05, **p<0.01, ***p<0.001, being considered as significant.

Results

3.1—Neuropilin Antagonist 47 (NRPa-47) Inhibits VEGF-R1/-R2 Phosphorylation.

We previously described a neuropilin antagonist (NRPa) so-called compound-1 that inhibited VEGF-A₁₆₅/NRP-1 binding, tumor survival and tumor growth in vivo, which is renamed here NRPa-47 [15]. However, the NRPa-47 mechanism of action remained elusive thus we focus our present report to describe its function and regulation of cell signaling. As NRP-1 interacts with both VEGF-R1 and VEGF-R2 in presence of VEGF-A₁₆₅ to mediate intrinsic tyrosine kinase activity, we first studied VEGF-R1/VEGF-R2 phosphorylation status in the presence of NRPa-47 at the half maximal inhibitory anti-proliferative concentration previously reported on MDA-MB-231 (IC₅₀=0.6+/−0.03 μM) [15]. Here, we showed that NRPa-47 significantly decreased tyrosine phosphorylation of both VEGF-R1 (20 to 40%) and VEGF-R2 (40 to 45%) since 5 min to 60 min on MDA-MB-231 (data not shown). Of note, this inhibition is not due to intrinsic kinase activity inhibition as previously reported [15]. To strengthen this result, we followed the expression of both HIF-1α and VEGF-A₁₆₅ mRNA as a negative feedback loop to validate the efficient abrogation of VEGF-R1 and VEGF-R2 phosphorylation mediated by NRPa-47. As expected, both HIF-1α and VEGF-A₁₆₅ mRNA are reduced in the presence of its antagonist in a time dependent manner with a maximum effect at 60 minutes (data not shown).

3.2—NRP-1/VEGF-Rs Downstream Signaling Modulation Induced by NRPa-47.

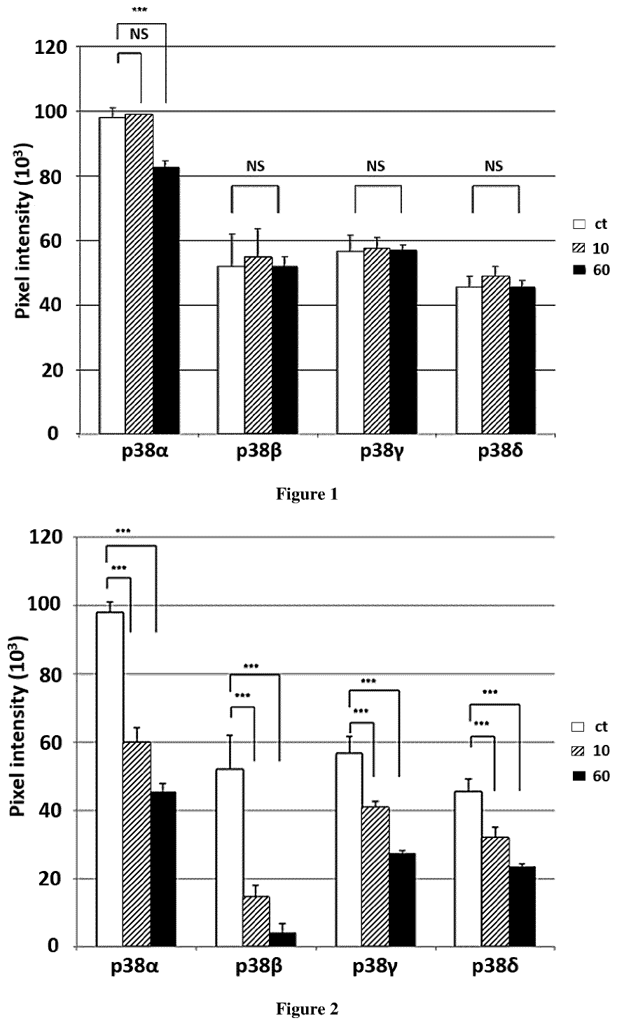

To better understand the effect of NRPa-47 on MDA-MB-231, we extended our study using biochemistry membrane platform targeting Mitogen-activated protein kinase (MAPK) and downstream kinases (data not shown). Surprisingly, NRPa-47 did not negatively modulate MAPK such as Extracellular signal-regulated kinases (ERK-1/-2) and c-Jun N-terminal kinases (JNK-1/-2/-3/-pan) pathways but contributed to their significant hyper-phosphorylations at 10 minutes and at 60 minutes of drug exposure, respectively (data not shown). These MAPK hyper-phosphorylations were in accordance with an increase in phosphorylation of their downstream substrates such as p90 ribosomal S6 kinase (RSK-2) observed at 60 minutes (data not shown). However, RSK-1 remained unchanged (data not shown). Furthermore, phosphorylations of AKT-1/-2/-3/-pan were up-regulated as well as its downstream p70 ribosomal S6 kinase (p70S6K) (data not shown). In summary, even if the dephosphorylation of VEGF-R1/-R2 has been induced by NRPa-47, their downstream kinases became hyper-phosphorylated. In front of these intriguing results, we focused our attention on the third MAPK which consists in the p38 pathway including (p38α, p38β, p38γ, p38δ). In this pathway, no significant variation of p38 phosphorylation has been observed except for p38α, which is significantly decreased at 60 minutes (FIG. 1). p38α downstream substrates, including small heat shock protein 27 (HSP27) and mitogen- and stress-activated protein kinase 2 (MSK2), were consequently dephosphorylated (data not shown). In addition, only the GSK-3β phosphorylation was affected (data not shown) due to the p38α phosphorylation defect but not to the ERK/AKT pathways. Taken together, NRPa-47 induces phosphorylation of ERK, JNK and AKT pathways but inhibits p38α phosphorylation and its downstream kinases.

3.3—Modified-NRPa-47 (NRPa-48) and its Own Effect on NRP-1/VEGF-Rs Downstream Signaling.

Face to these intriguing results between dephosphorylation of VEGF-R and hyper-phosphorylation of downstream signaling mediated by NRPa-47, we performed a new structural docking analysis. In this aim, we used the NRP-1 b1 domain defined-pocket by the tuftsin docking (data not shown). As we can note, the methyl group of the docked NRPa-47 is located outside the pocket (data not shown) and may constraint geometrically of the unconventional carboxythiourea linker in an unexpected way. Thus, we decided to remove this methyl group in order to study this structurally-related new compound called here NRPa-48 (data not shown). The ability of NRPa-48 to inhibit phosphorylation of both VEGF-R1 and VEGF-R2 has been also investigated at the half maximal inhibitory anti-proliferative concentration previously reported on MDA-MB-231 (IC₅₀=0.4+/−0.2 μM) [16]. As expected, NRPa-48 significantly exerted a tyrosine VEGF-R1 and VEGF-R2 kinases inhibitory capacity (data not shown) but with a lower efficiency than NRPa-47 (data not shown). Despite this fact, NRPa-48 is also efficient than NRPa-47 (0.6 vs 0.4 μM) to block MDA-MB-231 proliferation [16]. Thus, these results prompted us to investigate its role on VEGF-Rs downstream signaling.

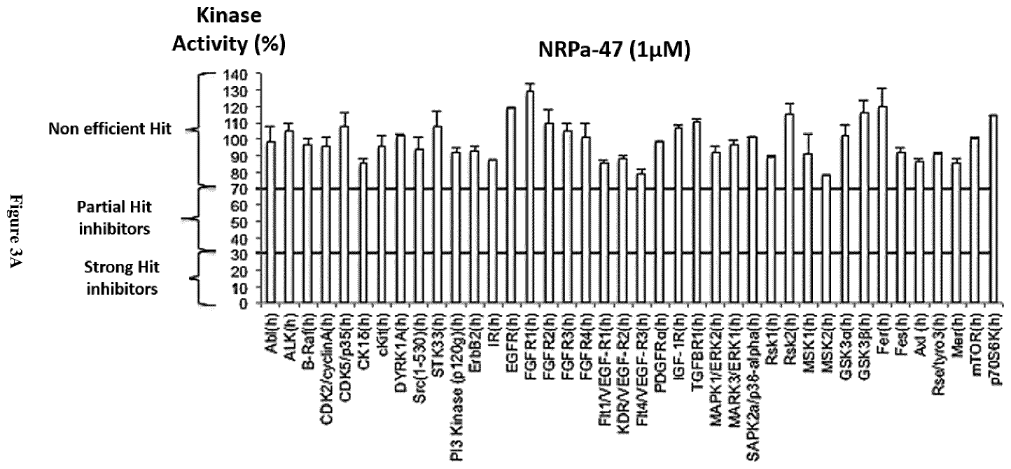

In contrast to NRPa-47, NRPa-48 significantly inhibited all tested MAPK (data not shown) such as ERK-1/-2, (data not shown), JNK-1/-2/-3/pan (data not shown), p38α/p38β/p38γ/p386 (FIG. 2) in a time dependent manner. Their own respective downstream kinase substrates such as RSK-1/RSK-2, MSK-2, HSP-27, GSK-3a/0 were also inhibited in a time dependent manner (data not shown). In addition, AKT pathway including AKT-1/-2/-3/-pan was also inhibited from 10 minutes until 60 minutes (data not shown). Taking together, methyl group removal from the NRPa-47 chemical structure conferred to NRPa-48 an intriguing efficiency to block activity of all MAPK and downstream kinases studied, induced by VEGF-A₁₆₅.

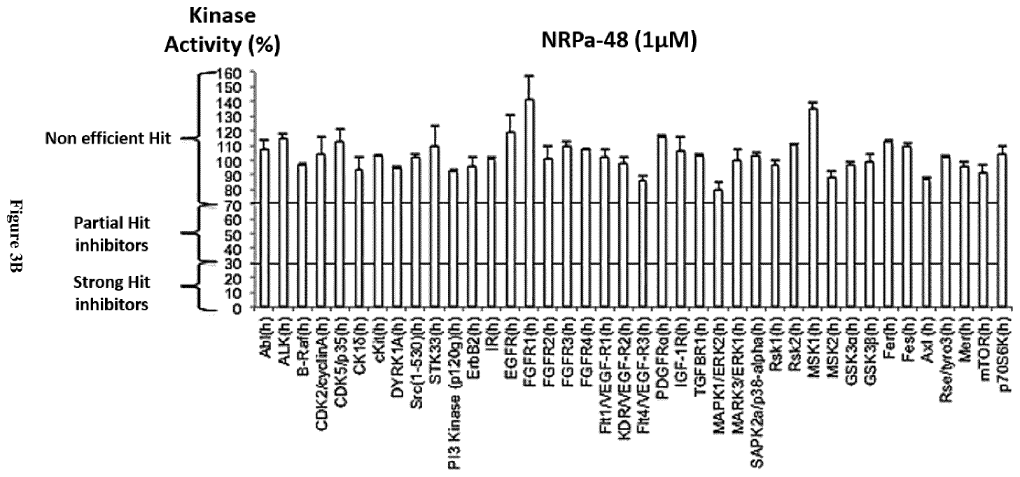

To strengthen the specificity of our hits, the activity of NRPa-47 and NRP-48 was evaluated in vitro on 40-selected kinases among growth factor receptors, cell cycle kinases, insulin receptors, etc. None of these were significantly affected by both hits since kinase activity results remain confined in the non-efficient Hits analysis section (FIGS. 3A-3B).

Particularly, no kinase activity decreases of NRP co-receptors such as VEGF-R1/-R2/-R3, TGF-ß1-R1, FGF-R1/-R2/-R3/-R4, EGF-Rs or on VEGF-Rs down-stream signaling has been observed (FIGS. 3A-3B). More importantly, none of the in vitro tested-kinase activity is blocked by these hits, in contrast to the observed kinase phosphorylation modulation induced by these hits in treated-tumor cells. Thus, this test reinforced the impact of these hits on the inhibition of the cancer therapeutic key factor p38α, which is a specific down-stream consequence of this treatment.

3.4—Apoptotic Pathway Induced by NRPa-47.

As both NRPa-47 and NRPa-48 exerted differential regulation of MAPK phosphorylation, we investigated the influence of this differential effect on cell death. Thus, we focused our study on NRPa induced apoptosis cascades. To unravel this mechanism of action, we performed an apoptosis proteome array experiment at a short (60 minutes) and long (48 hours) time (data not shown). We first analyzed cell death receptors such as TRAIL-R1/DR4, TRAIL-R2/DR5, FAS/TNFSF6, TNF-R1/TNSFRSF1 and their adaptor protein FADD. All of these parameters were drastically down-modulated since 60 minutes and lesser at 48 hours (data not shown). This result indicated that cell death induced by NRPa-47 seemed not be cell death receptors dependent. In addition, no pro-apoptotic proteins including Bad, Bax, SMAC/Diablo, HTRA2/Omi and cytochrome c have been induced by NRPa-47, in contrast a rapid decrease of these proteins was observed at short time drug exposure and remained decreased at the long time, excepted for the caspase-3 cleavage induction (data not shown). However, the anti-apoptotic proteins such as Bcl-2, Bcl-x, cIAP-1, cIAP-2, XIAP, Survivin, Livin, Clusterin as well as heat shock proteins (HSP-27, HSP-60, HSP-70) were significantly reduced since 60 minutes and remained decrease at 48 hours (data not shown).

Furthermore, NRPa-47 globally induced reduction of cell cycle protein expression such as p21/CIP1/CDNK1A, p27/kip1 and phosphorylation of Rad-17 (data not shown). Moreover, all phosphorylation sites of p53 protein were inhibited since 60 minutes (data not shown). Surprisingly, NRPa-47 induced rapid oxidative stress revealed by catalase induction but not the Serum paraoxonase/arylesterase 2 (PON-2) at 60 minutes (data not shown).

In conclusion, NRPa-47 induced down-modulation of pro-apoptotic and anti-apoptotic proteins as well as cell death receptors. However, NRPa-47 rapidly provoked an oxidative stress reflected by the catalase induction. In addition, expression of the inducible (HO-1/HMOX1/HSP32) and the constitutive (HO-2/HMOX2) heme oxygenase forms were both reduced since 60 minutes (data not shown). The cell death might be due to the decrease of both HIF-1α and survivin (data not shown).

3.5—Mechanism of NRPa-48-Induce Cell Death.

To clarify the opposite effect of NRPa-48 compared to NRPa-47 on MAPK regulation, we extended our study to unravel its mechanism of action on the apoptotic pathway (data not shown). In contrast to early NRPa-47 effect, NRPa-48 exerted a late effect on the regulation of apoptosis pathway as it was observed at 48 hours and not at 60 minutes (data not shown). In details, pro-apoptotic proteins such as Bad, Bax, SMAC/Diablo, HTRA2/Omi and cytochrome c were significantly decreased excepted for the caspase-3 cleavage induction (data not shown). The anti-apoptotic proteins such as Bcl-2, Bcl-x, cIAP-1, cIAP-2, XIAP, Survivin, Livin, Clusterin, the heat shock proteins (HSP-27, HSP-60, HSP-70) as well as the cell death receptors such as TRAIL-R1/DR4, TRAIL-R2/DR5, FAS/TNFSF6, TNF-R1/TNSFRSF1A and their adaptor protein FADD were also down-modulated (data not shown). NRPa-48 induced reduction of cell cycle protein expressions such as p21/CIP1/CDNK1A, p27/kip1, phosphorylation of Rad-17 and claspin (data not shown). In addition, all phosphorylation sites of p53 protein were inhibited (data not shown). In contrast to NRPa-47, NRPa-48 induced late oxidative stress detected by high level of catalase, however PON-2 level remained unchanged (data not shown). Interestingly, NRPa-48 has the capacity to inhibit HIF-1α expression as previously observed for NRPa-47 (data not shown). In addition, expression of the inducible (HO-1/HMOX1/HSP32) and the constitutive (HO-2/HMOX2) heme oxygenase forms were both reduced at 48 hours (data not shown).

Taken together, both NRPa exerted similar down-modulation of proteins involved in apoptosis and induced oxidative stress. More interestingly, the most important proteins modulated in this pathway by NRPa were HO-1/HMOX1/HSP32, survivin and HIF-1α.

In conclusion, even if NRPa-47 and NRPa-48 did not have the same effect on MAPK signaling, both conduct treated-cell to cell death program in similar manner but with a different timing.

3-6 In Vivo Anti-Tumor Activity of NRPa-48 on Xenografted-NOG Mice.

In vivo experiments were performed using NRPa-48 on MDA-MB-231-xenografted NOG mice to compare its efficiency on tumor growth inhibition to NRPa-47, which was previously described by our team in (PMID 24752068). One group was treated by force-feeding with NRPa-48 at 50 mg/kg three times a week and the remaining group was a negative control. Interestingly, the treated animals did not show any loss of weight suggesting that, at this concentration, NRPa-48 exhibits no acute toxicity (data not show). At Day-38 (21 days after starting treatment), tumor growth was strongly reduced by NRPa-48 (data not shown) since the size of the tumor was reduced by approximately 29% compared to the reference group (***p<0.001). The 50 mg/kg group remained largely efficient at Day-45 to exhibit a 34% tumor size reduction (data not shown). More interestingly, survival significantly increased when mice were treated with NRPa-48 at 50 mg/kg compared to the control group (data not shown). Thus, median survival was 35 days for the animals in the control group and over 56 days for the animals treated with NRPa-48 (p=0.008) (data not shown). Taking together, in vivo tumor growth inhibition mediated by NRPa-48 is efficient and 62% of treated mice at the end of treatment remained alive.

### Discussion

The development of NRPa brought new tools for cancer treatment and the knowledge of biochemical pathways involved in this process. In this report, we observed that a very small structural change in the structure of two structurally-related NRPa (NRPa-47 and NRPa-48), namely the suppression of a methyl group, induced major changes in the nature of the affected signaling pathways. NRP-1 inhibitors rapidly inhibit the HIF-1α protein and mRNA expression as well as the VEGF mRNA and thus may contribute to create an interference with the autocrine HIF-1α/VEGF feedback loop. This result is very intriguing since HIF-1α is an important cancer drug target [22]. In this context, NRPa may induce tumor cell starvation for VEGF and compromise their growth and survival. In addition, high oxidative stress reflected by the increase of catalase expression is induced at 60 minutes for NRPa-47 and at 48 h for NRPa-48. Even if both NRPa are capable to induce dephosphorylation of VEGF-Rs tyrosine kinase, their downstream targets are not similarly affected. The main difference observed for the two NRPa is restraint to the MAPK (ERK, INK, p38 except p38α) regulation which phosphorylations are increased by NRPa-47 and decreased by NRPa-48 (data not shown). Similar difference is observed on AKT pathway and its downstream target (p70S6kinase). Future investigations are needed to identify and/or clarify the alternative downstream pathway mediated MAPK phosphorylation in this context.

Nevertheless, this opposite effect on MAPK regulation conducting to hyper-phosphorylation as well as dephosphorylation may lead to tumor cell death. No significant apoptotic pathways emerged between both even if the down-modulation of death receptors, pro-apoptotic and anti-apoptotic proteins may occur an apoptotic/survival imbalance, which may also conduct to cell death. The most pivotal events reliable to the apoptosis induction are the decrease of survivin expression, the induction of oxidative stress (catalase), the down-regulation of heme oxygenase and the down-modulation of HIF-1α. Other HIF-1α inducer such as HO-1/HMOX1/HSP32, expression was inhibited as well as the p38α phosphorylation, which is also an activator of HO-1. The p38α pathway inhibition occur the p53 dephosphorylation, the defect of HSP27, GSK-38 and MSK2 phosphorylation as well as the down-modulation of survivin (data not shown). Disruption of survivin expression leads to increase apoptosis and decrease tumor growth.

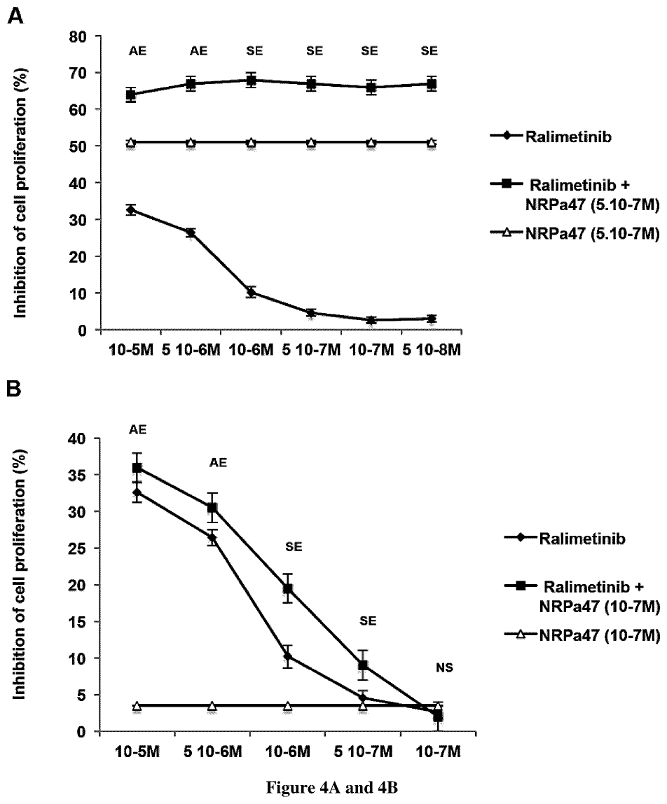

Taken together, this report highlighted the pivotal role of p38α in the cell signaling cascade mediated by NRPa. Several studies report p38α as drug target to develop specific inhibitors to treat cancer rely on p38 MAPK activity for progression. The association of p38α inhibitors with DNA-damaging chemotherapy may trigger cancer cell death by the impairment of p38α-mediated cell cycle arrest and DNA repair mechanisms [23]. Moreover, p38α inhibitors increase tumor cell sensitivity to chemotherapy such as 5-fluorouracil (5-FU) and Oxaliplatin® [24]. In this regard, the association of both NRPa with 5-FU® or Oxaliplatin® on breast cancer cells increased as expected their sensitivity to these respective drugs (data not shown). More importantly, the association of NRPa with Ralemitininb® (P-p38α inhibitor) strengthened this hypothesis since additional and/or synergistic effect of these drugs (depending of the dose used) significantly reduced breast cancer cell proliferation (FIGS. 4A-4B). In summary, NRPa might be used alone or in association with a drug to treat cancer. This observation brought newest interest for the development of NRPa.

### REFERENCES

Throughout this application, various references describe the state of the art to which this invention pertains. The disclosures of these references are hereby incorporated by reference into the present disclosure.
[1] Q. Pan, Y. Chanthery, W. C. Liang, S. Stawicki, J. Mak, N. Rathore, R. K. Tong, J. Kowalski, S. F. Yee, G. Pacheco, S. Ross, Z. Cheng, J. Le Couter, G. Plowman, F. Peale, A. W. Koch, Y. Wu, A. Bagri, M. Tessier-Lavigne and R. J. Watts, Blocking neuropilin-1 function has an additive effect with anti-VEGF to inhibit tumor growth, Cancer Cell 11 (2007) 53-67.
[2] A. L. Kolodkin, D. V. Levengood, E. G. Rowe, Y. T. Tai, R. J. Giger, D. D. Ginty, Neuropilin is a semaphorin III receptor, Cell 90 (1997) 753-62.
[3] S. Koch, S. Tugues, X. Li, L. Gualandi, L. Claesson-Welsh, Signal transduction by vascular endothelial growth factor receptors, Biochem. J. 437 (2011) 169-83.
[4] G. J. Prud'homme and Y. Glinka, Neuropilins are multifunctional coreceptors involved in tumor initiation, growth, metastasis and immunity, Oncotarget 3 (2012) 921-39.
[5] J. R. Wild, C. A. Staton, K. Chapple and B. M. Corfe, Neuropilins: expression and roles in the epithelium, Int. J. Exp. Pathol. 93 (2012) 81-103.
[6] Y. Xin, S. Bai, L. A. Damico-Beyer, D. Jin, W. C. Liang, Y. Wu, F. P. Theil, A. Joshi, Y. Lu, J. Lowe, M. Maia, R. K. Brachmann and H. Xiang, Anti-neuropilin-1 (MNRP1685A): unexpected pharmacokinetic differences across species, from preclinical models to humans, Pharm. Res. 29 (2012) 2512-21.
[7] M. Caunt, J. Mak, W. C. Liang, S. Stawicki, Q. Pan, R. K. Tong, J. Kowalski, C. Ho, H. B. Reslan, J. Ross, L. Berry, I. Kasman, C. Zlot, Z. Cheng, J. Le Couter, E. H. Filvaroff, G. Plowman, F. Peale, D. French, R. Carano, A. W. Koch, Y. Wu, R. J. Watts, M. Tessier-Lavigne and A. Bagri, Blocking neuropilin-2 function inhibits tumor cell metastasis, Cancer Cell 13 (2008) 331-42.
[8] A. Starzec, P. Ladam, R. Vassy, S. Badache, N. Bouchemal, A. Navaza, C. H. du Penhoat and G. Y. Perret, Structure-function analysis of the antiangiogenic ATWLPPR peptide inhibiting VEGF(165) binding to neuropilin-1 and molecular dynamics simulations of the ATWLPPR/neuropilin-1 complex, Peptides 28 (2007) 2397-402.
[9] G. Y. Perret, A. Starzec, N. Hauet, J. Vergote, M. Le Pecheur, R. Vassy, G. Leger, K. A. Verbeke, G. Bormans, P. Nicolas, A. M. Verbruggen and J. L. Moretti, In vitro evaluation and biodistribution of a 99mTc-labeled anti-VEGF peptide targeting neuropilin-1, Nucl. Med. Biol. 31 (2004) 575-81.
[10] A. Novoa, N. Pellegrini-Moise, D. Bechet, M. Barberi-Heyob and Y. Chapleur, Sugar-based peptidomimetics as potential inhibitors of the vascular endothelium growth factor binding to neuropilin-1, Bioorg. Med. Chem 18. (2010) 3285-98.
[11] H. Jia, A. Bagherzadeh, B. Hartzoulakis, A. Jarvis, M. Lohr, S. Shaikh, R. Agil, L. Cheng, M. Tickner, D. Esposito, R. Harris, P. C. Driscoll, D. L. Selwood and I. C. Zachary, Characterization of a bicyclic peptide neuropilin-1 (NP-1) antagonist (EG3287) reveals importance of vascular endothelial growth factor exon 8 for NP-1 binding and role of NP-1 in KDR signaling, J. Biol. Chem. 281 (2006) 13493-502.
[12] C. Nasarre, M. Roth, L. Jacob, L. Roth, E. Koncina, A. Thien, G. Labourdette, P. Poulet, P. Hubert, G. Crémel, G. Roussel, D. Aunis, D. Bagnard, Peptide-based interference of the transmembrane domain of neuropilin-1 inhibits glioma growth in vivo, Oncogene 29 (2010) 2381-92.
[13] A. Jarvis, C. K. Allerston, H. Jia, B. Herzog, A. Garza-Garcia, N. Winfield, K. Ellard, R. Agil, R. Lynch, C. Chapman, B. Hartzoulakis, J. Nally, M. Stewart, L. Cheng, M. Menon, M. Tickner, S. Djordjevic, P. C. Driscoll, I. Zachary and D. L. Selwood, Small molecule inhibitors of the neuropilin-1 vascular endothelial growth factor A (VEGF-A) interaction, J. Med. Chem. 53 (2010) 2215-26.
[14] A. Starzec, M A. Miteva, P. Ladam, B. O. Villoutreix, G. Y. Perret, Discovery of novel inhibitors of vascular endothelial growth factor-A-Neuropilin-1 interaction by structure-based virtual screening, Bioorg. Med. Chem. 22 (2014) 4042-8.
[15] L. Borriello, M. Montès, Y. Lepelletier, B. Leforban, W Q. Liu, L. Demange, B. Delhomme, S. Pavoni, R. Jarray, J L. Boucher, S. Dufour, O. Hermine, C. Garbay, R. Hadj-Slimane, F. Raynaud, Structure-based discovery of a small non-peptidic Neuropilins antagonist exerting in vitro and in vivo anti-tumor activity on breast cancer model, Cancer Lett. 349 (2014) 120-7.
[16] WQ. Liu, V. Megale, L. Borriello, B. Leforban, M. Montès, E. Goldwaser, N. Gresh, J P. Piquemal, R. Hadj-Slimane, O. Hermine, C. Garbay, F. Raynaud, Y. Lepelletier, L. Demange, Synthesis and structure-activity relationship of non-peptidic antagonists of neuropilin-1 receptor, Bioorg. Med. Chem. Lett. 24 (2014) 4254-9.
[17] R. Jarray, S. Pavoni, L. Borriello, B. Allain, N. Lopez, S. Bianco, W Q. Liu, D. Biard, L. Demange, O. Hermine, C. Garbay, F. Raynaud, Y. Lepelletier, Disruption of phactr-1 pathway triggers pro-inflammatory and pro-atherogenic factors: New insights in atherosclerosis development, Biochimie 118 (2015) 151-61.
[18] R. Jarray, B. Allain, L. Borriello, D. Biard, A. Loukaci, J. Larghero, R. Hadj-Slimane, C. Garbay, Y. Lepelletier, F. Raynaud, Depletion of the novel protein PHACTR-1 from human endothelial cells abolishes tube formation and induces cell death receptor apoptosis. Biochimie 93 (2011) 1668-75.
[19] C. W. Vander Kooi, M. A. Jusino, B. Perman, D. B. Neau, H. D. Bellamy and D. J. Leahy, Structural basis for ligand and heparin binding to neuropilin B domains. Proc. Natl. Acad. Sci. U.S.A. 104 (2007) 6152-7.
[20] A. N. Jain, Surflex: fully automatic flexible molecular docking using a molecular similarity-based search engine, J. Med. Chem. 46 (2003) 499-511.
[21] R. Abagyan and M. Totrov, Biased probability Monte Carlo conformational searches and electrostatic calculations for peptides and proteins, J. Mol. Biol. 235 (1994) 983-1002.
[22] A. R. Martin, C. Ronco, L. Demange and R. Benhida, Hypoxia inducible factor down-regulation, cancer and cancer stem cells (CSCs): ongoing success stories (2017), Med. Chem. Commun. under press. DOI: 10.1039/C6MD00432F.
[23] H. C. Reinhardt, A. S. Aslanian, J. A. Lees, M. B. Yaffe, p53-deficient cells rely on ATM- and ATR-mediated checkpoint signaling through the p38MAPK/MK2 pathway for survival after DNA damage, Cancer Cell 11 (2007) 175-89.
[24] A. A. Vaidya, M. B. Sharma, V. P. Kale, Suppression of p38-stress kinase sensitizes quiescent leukemic cells to anti-mitotic drugs by inducing proliferative responses in them, Cancer Biol. Ther. 7 (2008) 1232-40.

1. A method of treating cancer in a patient in need thereof comprising administering to the patient a therapeutically effective combination comprising at least one neuropilin antagonist and at least one p38α-kinase inhibitor.

2. The method of claim 1 wherein the subject is a human.

3. The method of claim 1 wherein the subject is a non-human mammal.

4. The method of claim 1 wherein the cancer is a hematopoietic or a non-hematopoietic cancer.

5. The method of claim 1 wherein the cancer is breast cancer.

6. The method of claim 1 wherein the cancer is triple-negative breast cancer.

7. The method of claim 1 wherein the cancer is neuropilin positive.

8. The method of claim 1 wherein the at least one neuropilin antagonist is selected from the group consisting of antisense polynucleotides, interfering RNAs, catalytic RNAs, RNA-DNA chimeras, neuropilin-specific aptamers, anti-neuropilin antibodies, neuropilin-binding fragments of anti-neuropilin antibodies, neuropilin-binding small molecules, neuropilin-binding peptides, and polypeptides that specifically bind neuropilin, such that the interaction between the neuropilin antagonist and neuropilin results in a reduction or cessation of neuropilin activity or expression.

9. The method of claim 1 wherein the at least one neuropilin antagonist inhibits the interaction between a neuropilin protein and a binding partner of the neuropilin protein.

10. The method of claim 1 wherein the at least one neuropilin antagonist is an antibody that specifically binds to a neuropilin and neutralizes its activity to activate neuropilin signalling pathway.

11. The method of claim 1 wherein the at least one neuropilin antagonist is NRPa-47 or NRPa-48.

12. The method of claim 1 wherein the at least one p38α-kinase inhibitor is selected from the group consisting of antisense polynucleotides, interfering RNAs, catalytic RNAs, RNA-DNA chimeras, p38-α-specific aptamers, anti-p38α antibodies, p38α-binding fragments of anti-p38α antibodies, p38α-binding small molecules, p38α-binding peptides, and polypeptides that specifically bind p38α, such that the interaction between the at least one p38α-kinase inhibitor and p38α results in a reduction or cessation of p38α kinase activity or expression.

13. The method of claim 1 wherein the at least one p38α-kinase inhibitor is selected from the group consisting of ARRY-371797, ARRY-614, AZD-7624, ralimetinib, LY-3007113, FX005, GSK610677, GW856553, SB-681323, KC706, UR-13870, PF-03715455, VX-745, SCID-469, PH-797804, VX-702, SB-202190, SB-203580, SB-239063, BIRB-796, BMS-582949, and pamapimod.

14. The method of claim 1 wherein the at least one neuropilin antagonist is NRPa-47 and the at least one p38α-kinase inhibitor is Ralimetinib.

15. The method of claim 1 wherein the at least one neuropilin antagonist is NRPa-48 and the at least one p38α-kinase inhibitor is Ralimetinib.

16. The method of claim 9 wherein the neuropilin protein is NRP-1.

17. The method of claim 16, wherein the binding partner of the neuropilin protein is VEGF-A₁₆₅.

18. The method of claim 10 wherein the neuropilin protein is NRP-1 or NRP-2).

19. The method of claim 18, wherein the at least one neuropilin antagonist inhibits the binding of the neuropilin protein and VEGF-A₁₆₅.
